(19) United States
(12) Patent Application Publication
(10) Pub. No.: US 2011/0011409 A1
(43) Pub. Date: Jan. 20, 2011

(54) Titspamm

(76) Inventor: Garrick Atkins, Benton Harbor, MI (US)

Correspondence Address: GARRICK ATKINS, P.O. box 9030, Benton Harbor, MI, 49023, US

(21) Appl. No.: 12/460,333

(22) Filed: Jul. 17, 2009

(51) Int. Cl. A61F 11/14

(52) U.S. Cl. 128/866

(57) ABSTRACT

Provided is a new and unmatched solution to preventing ear pain caused during the ascension and descend part of air travel. This product is one of a kind and is very unique. It is an air tight ear capping device called TITSPAMM.

### CROSS-REFERENCE TO RELATED APPLICATIONS

Not applicable

### STATEMENT REGARDING FEDARALLY SPONSORED RESEARCH OR DEVELOPMENT

Not applicable

### REFERENCE TO SEQUENCE LISTING

Not applicable

### BACKGROUND OF THE INVENTION

### Field of Invention

The present invention generally relates to devices or methods for the prevention of a devastating pain caused in the human ear by and from air travel during the ascending and descend part of the flight.

What has been tried and or what I have tried is the use of a device called an ear plug which has been designed to protect the ear from loud noise but was not designed for the prevention of water or air seeping into the ear canal which if too big will not fit properly or even if properly fitted may tend to fall out letting in water or the air that causes the ear pain in such an atmosphere during the ascension and descend part of air travel and or it may be to small wherein it could make its way further down the ear canal causing it to be very difficult and or painful to retrieve. In either way air seems to get into the ear canal and again air of such atmosphere causes pain while the plane is in either ascend or descend mode and the methods of chewing gum, drinking water, swallowing, hick upping and others during the ascension and descend of the flight which may seem to help out a little bit for some and not at all for many others leaving them left with unbearable pain and because of the pain and after the pain you are most likely left with a temporary loss of hearing that could last 24-72 hours or more depending on how many stops you have before you reach your destination and oh I cry, there must be a better way!

### BRIEF DESCRIPTION OF THE SEVERAL VIEWS OF THE DRAWING

Not Applicable

### DETAILED DESCRIPTION OF THE PREFERED EMBODIMENT

First and foremost it is pointed out that there are no drawings in this application because in this section, the invention will be explained along with the process of making and using this invention in such a full, clear, concise and exact terms so that any person of ordinary skill in the pertinent art, science or area could make and use the invention without any drawings, nor extensive experimentation.

This invention is the method of capping the ear air tight by the use of an air tight ear capping device make to prevent ear pain during air travel and is to be used while traveling on an airplane during the ascend and descend part of the flight, so that no air seeps or gets into the ear canal.

To simply make this simple product all you need to do is:

1. Cap the human ears with a concave vessel or bowl shaped object made from either materials such as but not limited to metal, glass, wood, rubber or plastic.

2. Then take of either such said materials in instruction 1 and simply attach or make to attach a strong flexible head band or any such device that will hold the said concave vessel in place so that the ears will be capped tight enough so that no air gets into the ear canal.

### SUMMARY OF THE INVENTION

The present invention is the method of capping the ear air tight and with the use of an air tight ear capping device. This device prevents ear pain during air travel. It is an object of this invention to provide an air tight ear pain prevention device that only needs to be worn during the ascend and descend part of the flight. A further object of this invention is to prevent the air of such atmosphere from getting into the ear canal, for without such air there will be no ear pain during the ascension and descend part of the flight. Still another object of the invention is to provide an air tight ear pain prevention device that will not only prevent ear pain but because of the temporary loss of hearing that comes during or after the pain will also be prevented. Still another object of the invention is so that the user may be hands free, device free and free from using and struggling with other devices and methods to prevent ear pain so that the user may enjoy the flight without pain and irritation and be able to hear normally after the trip. Thus the object of this invention and the use of this invention will leave the user living a better quality of life after such a trip. Therefore the user will be ready to take another such trip when needed and therefore many that do not take such trips anymore because of having had such a terrible experience with ear pain during such a flight or those who only take such trips while trying to fight the ear pain because of an emergency will now start taking again flight trips more frequently. Thus saving money at the gas pump from having to drive such long trips and this will help economize the Economy.

### DRAWINGS

Not applicable, not necessary, Note: see DETAILED DESCRIPTION OF THE EMBODIMENT.

What is claimed is:

1. The method of capping the ear air tight.

2. An air tight ear capping device.

3. The air tight ear capping device of claim 2 wherein said device is comprised of materials such as but not limited to metal, glass, wood, rubber or plastic.
